(19) United States
(12) Patent Application Publication
(10) Pub. No.: US 2012/0040970 A1
(43) Pub. Date: Feb. 16, 2012

(54) INTRANASAL DELIVERY SYSTEM FOR DANTROLENE

(76) Inventor: Abeer M. Al-Ghananeem, Lexington, KY (US)

(21) Appl. No.: 13/266,358

(22) PCT Filed: Apr. 26, 2010
(86) PCT No.: PCT/US10/32375

§ 371 Date: Oct. 26, 2011

(30) Foreign Application Priority Data
Apr. 27, 2009 (US) 61/172982

(51) Int. Cl. A61K 31/4178; A61P 3/00; A61P 25/00; A61K 31/551; A61K 31/451

(52) U.S. Cl. 514/221; 514/390; 514/325

(57) ABSTRACT

The present invention provides a method for rapidly and reliably delivering dantrolene, or derivatives thereof, alone or in combination with other compounds, to the systemic circulation by administration via the nasal route to produce rapid onset of beneficial effects in the treatment or prevention of malignant hyperthermia (MH), spasticity, and Ecstasy intoxication. The present invention further provides intranasal pharmaceutical compositions comprising dantrolene sodium or any pharmaceutically acceptable salts thereof in a variety of pharmaceutical dosage forms, with and without other compounds.

### CROSS-REFERENCE TO RELATED APPLICATIONS

This application claims priority to U.S. Provisional Patent Application Ser. No. 61/172,982, which was filed on Apr. 27, 2009, the entirety of which is incorporated herein by reference for all purposes.

### FIELD OF THE INVENTION

The present invention provides a method for rapidly and reliably delivering dantrolene sodium, or any pharmaceutically acceptable salt thereof, alone or in combination with other compounds, to the systemic circulation by administration via the nasal route to produce rapid onset of beneficial effects in the treatment and prevention of malignant hyperthermia (MH) and/or spasticity. The present invention further provides intranasal pharmaceutical compositions comprising dantrolene and pharmaceutically acceptable salts thereof in a variety of unique pharmaceutical dosage forms, with and without other compounds.

### BACKGROUND OF THE INVENTION

Malignant hyperthermia (MH) is a hypermetabolic syndrome of skeletal muscle. It is characterized by cellular calcium increase and ATP depletion, and is mediated by exposure to general anesthetic agents. Incidence of MH is 1:10,000 anesthetic procedures and is characterized by muscle rigidity as an outstanding sign, added to heart arrhythmias and tachycardia. Patients may die during acute MH crisis or during follow up days. All general anesthesia settings should have 36 dantrolene standby vials ready for emergency administration by continuous rapid intravenous push beginning at a minimum dose of 1 mg/Kg, and continuing until symptoms subside or the maximum cumulative dose of 10 mg/Kg has been reached.

Despite more than 25 years of research, dantrolene is still the only currently available drug for the effective and specific treatment of MH crises in man. The principal disadvantages of dantrolene are its poor water solubility and difficulties in rapidly preparing a suitable solution for intravenous administration.

Reconstituting dantrolene intravenous dosage form fast enough to reverse malignant hyperthermia (MH) in an adult is a difficult task. Gronert et al. advised that this job usually requires the full-time efforts of three or four people.

Thus, in the emergency situation, although intravenous administration is probably the most rapid way of seizure or malignant hyperthermia suppression, an alternative and more convenient approach is highly needed when intravenous administration is not immediately available, for instance, because of the delay on transferring patient to hospital or delay in preparing the intravenous solution, or arriving of emergency medical personnel.

Furthermore, dantrolene is available in the form of oral capsules for the treatment of spasticity. Thus, the nasal administration can be used in some children and geriatric patients' population that are unable to swallow the dantrolene sodium capsules currently marketed for spasticity.

Systemic drug delivery through nasal route has been demonstrated as a possible route of administration and a promising approach for rapid-onset delivery of drugs to systemic circulation and central nervous system, owing to the extensive microcirculation underneath nasal mucosa and the preferential drug delivery to brain.

Therefore, in view of the aforementioned deficiencies attendant with prior art methods of treatment of MH and spasticity, it should be apparent that there still exists a need in the art for a rapid, reliable, safe and convenient method of delivering dantrolene in a patient in need of such treatment.

### BRIEF SUMMARY OF THE INVENTION

A major object of the present invention is to provide a method for safely and conveniently administering dantrolene to a patient in need of prevention or treatment of MH and/or spasticity. A rapid and reliable response is achieved. The method comprises the intranasal administration of an effective amount of dantrolene to a patient suffering from, or at risk for, MH and/or spasticity.

The object of the present invention is to provide a delivery system to enhance the rate of delivery of dantrolene to the systemic circulation by administering dantrolene via the nasal route in order to speed the onset of effect and reduce the dose required for its beneficial effect. Furthermore, the intranasal administration can be used in emergency cases until the intravenous dosage form is available.

Furthermore, the intranasal administration of dantrolene in patient that cannot swallow the capsule will not just improve patient compliance but also improve the drug bioavailability by direct absorption into the blood, thereby avoiding extensive first-pass metabolism which may significantly lower the plasma concentrations of dantrolene when it is administered via the oral route. As a result, small doses of dantrolene sodium, or any pharmaceutically acceptable salt thereof, can be administered which will result in fewer side effects, and the drug will be more tolerable and more effective in patients suffering from spasticity.

Intranasal dosage forms containing dantrolene in combination with other drugs used in the treatment of MH and/or spasticity may also be employed in the practice of the present invention. With the foregoing and other objects, advantages and features of the invention that will become hereinafter apparent, the nature of the invention is further explained in the following detailed description of the preferred embodiments of the invention and in the appended claims.

### BRIEF DESCRIPTION OF THE DRAWINGS

This invention, as defined in the claims, can be better understood with reference to the following drawings:

FIG. 1 illustrates the mean plasma dantrolene sodium levels following intranasal and intravenous administration at 1 mg/kg single dose in rabbits (n=3).

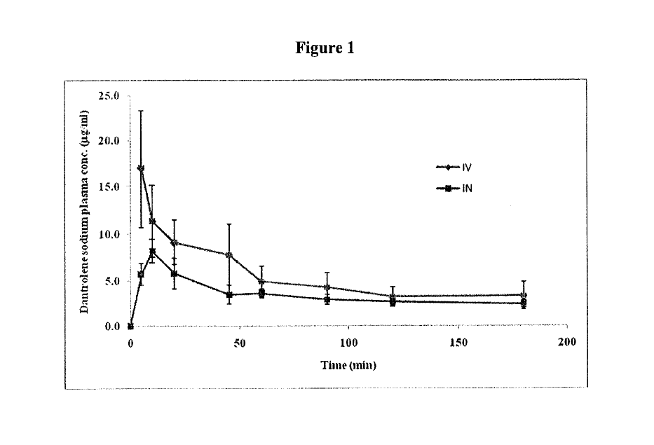

In the following description of the illustrated embodiments, references are made to the accompanying drawings, which form a part hereof, and in which is shown by way of illustration various embodiments in which the invention may be practiced. It is to be understood that other embodiments may be utilized and structural and functional changes may be made without departing from the scope of the present invention.

### DETAILED DESCRIPTION OF THE INVENTION

Thus, the present inventors have discovered a novel method for the delivery of dantrolene sodium, or any pharmaceutically acceptable salt thereof, to a patient in need of such treatment, comprising the intranasal administration of dantrolene sodium, or pharmaceutically acceptable salt thereof. This method offers significant clinical advantages and patient compliance over the prior art. More specifically, the inventors sought to provide a rapid, reliable, safe, effective and convenient treatment for administering dantrolene sodium, or any pharmaceutically acceptable salt thereof, to a patient in need of such treatment, which comprises the administration of dantrolene sodium, or any pharmaceutically acceptable salt thereof, intranasally, thus providing nearly instantaneous response for patient in need of such urgent treatment until the intravenous dosage form is available. By using the method of the present invention, which produces an instantaneous response, the drug will become more tolerable in patients who cannot swallow dantrolene capsules and more effective in treating patients suffering from MH until the intravenous form is made ready.

More particularly, the present invention concerns the intranasal administration of dantrolene, having the chemical structure of formula (I):

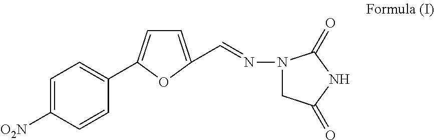

or dantrolene sodium or a pharmaceutically acceptable salt thereof, or a pharmaceutical composition intended for nasal administration comprising at least one of said compounds.

According to the present invention, dantrolene may be administered intranasally either as a free acid, or in the form of a pharmaceutically acceptable salt thereof.

In order to provide a clear and consistent understanding of the specification and claims, including the scope to be given such terms, the following definitions are provided:

The term “administration” of the pharmaceutically active compounds and the pharmaceutical compositions defined herein includes intranasal application.

The term “dantrolene” as used herein includes the free acid form of this compound as well as pharmaceutically acceptable salts thereof. In one embodiment, the pharmaceutical composition comprises dantrolene sodium; however, other pharmacologically acceptable salts thereof can be utilized as well.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

“Pharmaceutically acceptable salt” refers to pharmaceutically acceptable salts of dantrolene which salts are derived from a variety of organic and inorganic counter ions well known in the art and include, by way of example only, sodium, potassium, calcium, magnesium, ammonium, tetraalkylammonium, and the like.

The terms “effective amount” or “pharmaceutically effective amount” refer to a nontoxic but sufficient amount of the agent to provide the desired biological result. That result can be reduction and/or alleviation of the signs, symptoms, or causes of a disease, such as spasticity and malignant hyperthermia, or any other desired alteration of a biological system. Such amounts are described below. An appropriate “effective” amount in any individual case may be determined by one of ordinary skill in the art using routine experimentation.

As used herein, the term “excipient” means the substances used to formulate active pharmaceutical ingredients (API) into pharmaceutical formulations; in a preferred embodiment, an excipient does not lower or interfere with the primary therapeutic effect of the API. Preferably, an excipient is therapeutically inert and compatible. The term “excipient” encompasses carriers, diluents, vehicles, solubilizers, stabilizers, bulking agents, acidic or basic pH-adjusting agents and binders. Excipients can also be those substances present in a pharmaceutical formulation as an indirect or unintended result of the manufacturing process. Preferably, excipients are approved for or considered to be safe for human and animal administration, i.e., GRAS substances (generally regarded as safe). GRAS substances are listed by the Food and Drug administration in the Code of Federal Regulations (CFR) at 21 CFR 182 and 21 CFR 184, incorporated herein by reference.

By “compatible” herein is meant that the components of the compositions which comprise the present invention are capable of being commingled without interacting in a manner which would substantially decrease the efficacy of the pharmaceutically active compound under ordinary use conditions.

As used herein, the terms “formulate” refers to the preparation of a drug in a form suitable for administration to a mammalian patient, preferably a human. Thus, “formulation” can include the addition of pharmaceutically acceptable excipients, diluents, or carriers and pH adjusting agents.

The term “permeation enhancer” or “penetration enhancer” as used herein refers to an agent that improves the rate of transport of a pharmacologically active agent (e.g., dantrolene) across the nasal mucosal surface. Penetration enhancers, for example, increase the rate at which the pharmacologically active agent permeates through membranes and enters the bloodstream. An “effective” amount of a permeation enhancer as used herein means an amount that will provide a desired increase in mucosal membranes permeability to provide, for example, the desired rate of penetration of a selected compound, and amount of compound delivered.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Treating” or “treatment” of a disease includes: (1) preventing the disease, i.e., causing the clinical symptoms of the disease not to develop in a mammal that may be exposed to or predisposed to the disease but does not yet experience or display symptoms of the disease, (2) inhibiting the disease, i.e., arresting or reducing the development of the disease or its clinical symptoms, or (3) relieving the disease, i.e., causing regression of the disease or its clinical symptoms.

The intranasal composition of the present invention is useful in the treatment of a mammalian organism, including animals and humans.

According to the present invention, the term “patient” will encompass any mammal requiring treatment with dantrolene sodium, or any pharmaceutically acceptable salt thereof, particularly a human patient, suffering from MH or spasticity.

A still further aspect of this invention is a pharmaceutical composition of matter that comprises dantrolene sodium, or pharmaceutically acceptable salts thereof, and pharmaceutically acceptable carriers.

For therapeutic use in the prevention or treatment of MH or spasticity, dantrolene or salts thereof, can be conveniently administered in therapeutically effective amounts in the form of a pharmaceutical composition containing dantrolene, or a salt thereof, and a pharmaceutically acceptable intranasal carrier.

Typically, the carrier may be a powder, liquid, solution, spray, drops, gel, or combination thereof. In a preferred embodiment, the carrier is a pharmaceutically acceptable powder. Such compositions may require the use of one or more solubilizing agents to effect dissolution of the drug at the nasal mucosa. Suitable applications of solubilizing agents are exemplified below.

The preferred dosage forms are a nasal puff or spray, for rapid absorption and rapid onset of beneficial effects, and a nasal gel, typically for more prolonged effects.

Dantrolene sodium or any pharmaceutically acceptable salt may be formulated with the carrier into any desired unit dosage form. Unit dosage forms such as powders, solutions, suspensions, and water-miscible solids are particularly preferred.

Each carrier must be “acceptable” in the sense of being compatible with the other ingredients in the formulation and not injurious to the patient.

As used herein, a “pharmaceutically acceptable carrier” is a material that is nontoxic and generally inert and does not affect the functionality of the active ingredients adversely. Examples of pharmaceutically acceptable carriers are well known and they are sometimes referred to as diluents, vehicles or excipients. The carriers may be organic or inorganic in nature. In addition, the formulation may contain additives such as thickening or gelling agents, emulsifiers, wetting agents, buffers, stabilizers, and preservatives such as antioxidants.

To prepare formulations suitable for intranasal administration, powders, solutions and suspensions are sterilized and are preferably isotonic.

While it is possible for dantrolene sodium or any pharmaceutically acceptable salt to be administered a lone as a powder, it is preferably present as a micronized pharmaceutical formulation.

In a preferred embodiment dantrolene sodium or any pharmaceutically acceptable salt may be formulated with excipients such as; solvent vehicles, complexing agents, polymeric carrier, and cosolvents.

In a preferred embodiment the dantrolene is formulated with polymeric carrier. More preferably, dantrolene sodium or any pharmaceutically acceptable salt is formulated in microencapsulation formulations such as nano/microemulsion, and nanoparticles.

In a preferred embodiment, the formulation is applied in a powder form.

In another preferred embodiment, the dantrolene formulations can be applied to the nasal cavity in a liquid form.

The dosage of dantrolene sodium or pharmaceutically acceptable salts thereof in the compositions of the invention will vary depending on several factors, including, but not limited to, the age, weight, and species of the patient, the general health of the patient, the severity of the symptoms, whether the composition is being administered alone or in combination with other agents, the incidence of side effects and the like.

The desired dose may be administered as needed, and may be administered repeatedly over a period of months or years. Higher and lower doses may also be administered. A major advantage of the present invention is the extremely rapid onset of response, which enables the physician to adjust the dose to produce only the desired effects and nothing more, thereby optimizing drug use and minimizing side-effects.

The daily dose may be adjusted taking into account, for example, the above-identified variety of parameters. Typically, dantrolene may be administered in an amount of up to about 100 mg/dose. Preferably, the amount of dantrolene administered will not exceed 50 mg/dose. The typical range is 25 to 100 mg/dose, preferably 25 to 50 mg/dose. However, other amounts may also be administered. The dose is usually administered once or twice daily.

In particular, much smaller amounts of dantrolene will be required when administered intranasally, in accordance with the present invention.

Absorption of the drug from the pharmaceutical composition of the invention is rapid such that the drug reaches the systemic circulation almost as fast as through injection and appreciably faster than oral administration, which is highly advantageous for the rapid relief of MH or spasticity.

Furthermore, the water insolubility and any possible irritant properties of the active pharmaceutical ingredient are reduced by presenting the drug to the nasal mucosal membranes in the form of a cyclodextrin inclusion complex.

The present invention achieves these advantages by molecular encapsulation of the drug in a cyclodextrin, so forming a molecular inclusion complex which may be used in the solid form for the preparation of nasal inhalation powders (insufflations). The inclusion complex may be used in the liquid state for the preparation of metered dose sprays, drops or pressurized aerosols for nasal administration. The inclusion complexes of dantrolene may therefore be prepared according to methods known in the art such as spray drying, freeze drying and kneading.

Mucoadhesive polymers may be used to prolong the transient time and residence time in the nasal cavity. Typical mucoadhesive polymers could be used alone or in combination. In a preferred embodiment, mucoadhesive polymers include polyacrylate, cellulose, starch, and chitosan.

Penetration enhancers may be used to promote the passage of dantrolene across the mucosal membranes. Typical permeation enhancers could be used alone or in combination. In preferred embodiment, permeation enhancers include fatty acids and their salts such as sodium oleate, sodium laurate, and bile salts such as sodium glycodeoxycholate, sodium glycocholate, sodium cholate and sodium laurodeoxycholate. Other penetration enhancers may include tensides, ionic surfactants such as sodium lauryl sulphate, or non-ionic surfactants such as polyoxyethylene lauryl ethers, fusidates such as sodium taurodihydrofusidate. Other specific enhancers include azone and chitosan.

In a preferred embodiment, the concentrations of permeation enhancers are between 0.1% to 5%, more preferably the permeation enhancer concentration is between 0.25% to 3% by weight of the composition.

Liquid compositions suitable for nasal administration may contain a suitable quantity of viscosity modifying agents such as hypromellose or carbopol 934P and preservative agents such as benzalkonium chloride, chlorhexidine gluconate or thiomersal.

While it is possible for the active ingredient to be administered alone, as noted above, it is preferably present as a pharmaceutical formulation.

The formulations of the present invention comprise at least one active ingredient, as defined above, together with one or more acceptable carriers thereof and optionally other therapeutic agents.

The method of the present invention may be practiced by nasal administration of dantrolene by itself or in a combination with other active ingredients in a pharmaceutical composition. Other therapeutic agents suitable for use herein are any compatible drugs that are effective by the same or other mechanisms for the intended purpose, or drugs that are complementary to dantrolene. Such additional drugs include, but are not limited to, baclofen, benzodiazepines, cyproheptadine, tetrahydrocannabinol, and/or gabapentin.

The compounds utilized in combination therapy may be administered simultaneously, in either separate or combined formulations, or at different times than the dantrolene, e.g., sequentially, such that a combined effect is achieved. The amounts and regime of administration will be adjusted by the practitioner, by preferably initially lowering their standard doses and then titrating the results obtained. The therapeutic method of the invention may be used in conjunction with other therapies as determined by the practitioner.

Having now generally described this invention, the same will be better understood by reference to certain specific examples, which are included herein for purposes of illustration only and are not intended to be limiting of the invention or any embodiment thereof, unless so specified. Set forth below are examples of experimental procedures designed to demonstrate the features of this invention in mammalian models, and examples of pharmaceutical dosage forms that embody and illustrate its reduction to practice.

### EXAMPLES

The following Examples are provided to illustrate certain aspects of the present invention and to aid those of skill in the art in practicing the invention. These Examples are in no way to be considered to limit the scope of the invention in any manner.

### Example 1

Formulate by adding 50 mL of distilled water to 100 mg micronized dantrolene sodium, 0.3% w/w sodium lauryl sulfate, and 50 mL ethanol. Then, the solution was lyophilized to obtain a uniform lyophilized product. This product was put in a mortar and grounded to obtain a powdered preparation. The resulting powder preparation contained 100 mg of dantrolene sodium. This powder preparation is to be used as nasal administration powdered preparation.

### Example 2

Formulate by adding 100 mL of distilled water to 100 mg micronized dantrolene sodium, 0.3% w/w sodium laurodeoxycholate and 0.4% w/w carbopol 981. The resulting solution was heated at 50 degrees for 30 min. Then, the solution was cooled to room temperature and lyophilized to obtain a uniform lyophilized product. This product was put in a mortar and grounded to obtain a powdered preparation. The resulting powder preparation contained 100 mg of dantrolene sodium. This powder preparation is to be used as nasal administration powdered preparation.

### Example 3

Micronized dantrolene sodium (250 mg) and 2-hydroxypropyl-beta-cyclodextrin (100 mg) are mixed. Purified deionized water (4 mL) with a pH of 8 is added with vigorous kneading to form a uniform paste with optional heating. Kneading is continued for 30 minutes and the paste is freeze dried. The dried complex is grounded in a mortar to obtain uniform lyophilized powder containing 250 mg dantrolene sodium.

### Example 4

Micronized dantrolene sodium (100 mg) was dissolved in DMSO and ethanol mixture (50:50) and added to 20 mg of sodium starch glycolate. The components were mixed and then placed in a rotary evaporator. The mixture solvents were carefully evaporated at 35° C. and below 100 mBar until the powder were completely dry.

This product was put in a mortar and grounded to obtain a powdered preparation. The resulting powder preparation contained 100 mg of dantrolene sodium. This powder preparation is to be used as nasal administration powdered preparation.

### Example 5

Microencapsulation of dantrolene in chitosan nanoparticles for intranasal delivery. Chitosan nanoparticles were prepared based on the ionotropic gelation of chitosan with tripolyphosphate (TPP) anions. Chitosan was dissolved in purified water (pH 4.0) at 0.20% w/v. Nanoparticles were obtained upon the addition of a TTP aqueous solution (0.5 mL, 0.2% w/v in 0.1 N NaOH) to chitosan solution (2 mL, 0.2% w/v) under magnetic stirring at room temperature. For the association of dantrolene to chitosan nanoparticles, micronized dantrolene was dissolved in 0.01N NaOH (2 mg/mL) and then incorporated in the TPP solution. Nanoparticles loaded with dantrolene were obtained as described above. The concentration of dantrolene in the TPP solution was adjusted in order to obtain chitosan nanoparticles containing around 80% w/w of dantrolene.

Nanoparticles were concentrated by centrifugation at 8,000×g on a 10 μL glycerol bed, for 30 min. Supernatant was freeze dried and the nanoparticles were stored until use. For dosing, this resulting powder preparation is to be used as nasal administration powdered preparation. Alternatively, the nanoparticles could be resuspended in phosphate buffer (pH 6.4) and placed in a spray bottle for nasal administration.

### Example 6

Surfactant, co-surfactant mixture comprised Tween 80, polyethylene glycol 400, and ethanol was mixed in 1:1:1 ratio continuously using magnetic stirrer for 15 minutes until clarity of solution was observed. Micronized dantrolene sodium (200 mg) was added to 1 mL of sesame oil and vortexed for 3 minutes to form a suspension. The sesame oil mixture was added drop wise to 8 mL of surfactant co-surfactant mixture with continuous stirring and optional heating. Distilled water (1 mL) was then added gradually to dilute the formulation and the whole formulation was mixed at 500 rpm for 30 minutes.

### Example 7

### An In Vivo Absorption of Dantrolene Sodium From the Nasal Cavity

### Experimental Technique

The nasal absorption of dantrolene was investigated in vivo using male New Zealand albino rabbits weighing between 3.5-4.0 kg. Rabbits were acclimated for 2 weeks before the study. All research and testing activities related to this work were reviewed and approved by the Institutional Animal Care and Use Committee (IACUC) prior to the initiation of this research, and during its execution.

Following introduction of lidocaine local anesthesia, a catheter was placed in the marginal ear artery of the rabbit for blood sample collection. For intravenous administration, a catheter was placed in the marginal ear vein of the rabbit and dantrolene formulation (0.1 mL) was administered; a sterile drug solution was prepared by filtration (double 0.22 μfilters).

A comparative determination of the blood levels of dantrolene delivered both intravenously and nasally was carried out in a random cross-over design. Two weeks were allowed between treatments. Prior to intranasal administration, each spray device was primed by activating the pump ten times. The nasal formulations were administered by pumping a single spray into each nostril while the rabbits head was held in an upright position, such that a total dose of 1 mg/kg was administered. The actuator tip was inserted about 1 cm into the nostril and forehead of the rabbit and the rabbit was kept in the upright position for 2 min after drug administration to prevent leakage of the nasal formulation out of the nostril. In order to determine the absolute bioavailability of the nasal dose, rabbits received 1 mg/kg dantrolene intravenously injected into the marginal ear vein cannula followed by a 0.2 mL flush with 10% v/v heparin/normal saline solution to keep the cannula patent. Blood samples were collected from a 2-3 mm longitudinal venosection catheter of the marginal ear vein. This enabled easy and repeated sampling after washing out the blood clot. Blood samples (about 0.5 mL) were collected at baseline, before dantrolene dose administration; and subsequently at 5, 10, 20, 45, 60, 120 and 180 min post dose administration. Blood samples were collected by allowing the blood to drip freely from the marginal ear vein catheter into pre-heparinized tubes. Plasma (200 μL) was separated by centrifugation at 3000 rpm for 10 min, and was frozen at −20° C. in polypropylene tubes until the time of analysis.

### Extraction Procedure

Samples were left to thaw at room temperature. The samples were vortexed for 30 seconds and 100 μL aliquot parts were taken and placed in centrifuge tubes. 4 pH of HCl (1 N) added to each tube and mixed by aspirating and dispensing then vortexed for 2 minutes to get a pH of 3.

300 μL of 1-chlorobutane was added into the plasma aliquots followed by 100 μL of acetonitrile. The centrifuge tubes were then vortexed for 2 minutes and centrifuged at 10000 rpm for 15 min. 400 μL of the supernatant was transferred into newly labeled centrifuge tubes and allowed to evaporate on the Speed Vac Evaporator for 15 minutes. 50 μL of methanol:water (50:50) was used to reconstitute the totally evaporated samples, vortexed for another 2 min then transferred into HPLC vials (with inserts) for analysis.

Ultra Performance Liquid Chromatography with Mass Spectroscopy (HPLC/MS) Assay Method

Chromatography was performed on a Waters Acquity HPLC® BEH (2.1 mm×100 mm, 1.7 μm) RP18 column equipped with a VanGuard pre-column (Waters Acquity HPLC® BEH (2.1 mm×5 mm, 1.7 μm) RP18) with a mobile phase consisting of solvent A; methanol and solvent B; ammonium acetate (5 mM). The solvents gradient program ranges from 40% solvent A initially, to 65% over the next 4 minutes. Flow stays as is for another 2.5 minutes then the organic phase (mobile A) spikes to 90% over the next 30 seconds. Those percentages are maintained for 3 minutes to allow a thorough flushing of the column. By the 10^{th} minute of the run, the flow rate of the methanol goes back to the initial percentage of 40% over 1 minute which then stabilizes till the end of the run. The flow-rate was set at 0.15 mL/min with a total run time of 15 minutes. Selected ion monitoring (SIM) was performed in the positive mode, M+=315.08 m/z. The capillary voltage and cone voltage were maintained at 4.3 kV and 41 V, respectively. The source temperature and desolvation temperature were set at 60 and 350° C. Nitrogen was used as both the cone gas and the desolvation gas (600 L/h). Mass chromatograms and mass spectral data were acquired and processed by MassLynx software.

While this invention has been described as having a preferred embodiment, it is understood that the invention is not limited to the illustrated and described features. To the contrary, the invention is capable of further modifications, uses, and/or adaptations following the general principles of the invention and therefore includes such departures from the present disclosure as come within the known or customary practice in the art to which the invention pertains, and as may be applied to the central features set forth above, and which fall within the scope of the appended claims.

It would be obvious to those skilled in the art that modifications or variations may be made to the preferred embodiment described herein without departing from the novel teachings of the present invention. All such modifications and variations are intended to be incorporated herein and within the scope of the claims.

1. A method for preventing or treating MH and/or spasticity, said method comprising
intranasally administering to a patient in need of such prevention or treatment an effective amount of a pharmaceutical composition comprising dantrolene, or a pharmaceutically acceptable salt thereof;
wherein said pharmaceutical composition is intranasally administered; and
wherein said pharmaceutical composition is in the form of a powder.

2. (canceled)

3. An intranasal pharmaceutical composition suitable for the prevention or treatment of MH and/or spasticity, comprising an effective amount of dantrolene, or a pharmaceutically acceptable salt thereof along with a pharmaceutically acceptable nasal carrier; wherein said composition is in the form of a powder.

4. The composition according to claim 3, wherein the carrier is powder.

5. (canceled)

6. (canceled)

7. The method of claim 1, wherein the dantrolene is in the form of dantrolene free acid, dantrolene sodium, or any acceptable salt.

8. The method of claim 1, wherein the dantrolene may be administered practiced by nasal administration of dantrolene by itself or in a combination with other active ingredients in a pharmaceutical composition such as baclofen, benzodiazepines, cyproheptadine, tetrahydrocannabinol, and/or gabapentin.

9. A method of providing fast relief from the symptoms of MH or spasticity comprising nasal administration of dantrolene sodium or pharmaceutically acceptable salt to a subject in need thereof.

10. A method for rapidly and reliably delivering via nasal administration a therapeutically effective concentration of dantrolene to the systemic circulation of a patient for the treatment of MH and/or spasticity.
